(19) United States
(12) Patent Application Publication
(10) Pub. No.: US 2009/0126733 A1
(43) Pub. Date: May 21, 2009

(54) XENON RECOVERY FROM AMBIENT PRESSURE VENTILATOR LOOP

(75) Inventors: Sudhir S. Kulkarni, Wilmington, DE (US); Christian Daviet, Paris (FR)

Correspondence Address: AIR LIQUIDE;Intellectual Property, 2700 POST OAK BOULEVARD, SUITE 1800, HOUSTON, TX, 77056, US

(73) Assignees: L'Air Liquide Societe Anonyme Pour L'Etude Et L'Exploitation Des Procedes Georges Claude, Paris (FR); TAEMA, Antony (FR)

(21) Appl. No.: 12/126,644

(22) Filed: May 23, 2008

Related U.S. Application Data
(60) Provisional application No. 60/939,650, filed on May 23, 2007.

(51) Int. Cl. A61M 16/10; A61M 16/12; A61M 16/22; A61M 21/00; B01D 53/14

(52) U.S. Cl. 128/203.16; 128/203.12; 128/205.27; 128/205.28; 128/203.14; 128/910; 95/130; 95/138; 95/139

(57) ABSTRACT

Xe exhaled from a patient is recovered with a polymeric membrane.

### CROSS-REFERENCE TO RELATED APPLICATIONS

This patent application claims the benefit of U.S. Provisional Patent Application 60/939,650 filed May 23, 2007.

### BACKGROUND

Xenon is considered to be superior to standard anaesthetics because of its fewer side effects and quicker patient recovery. However, Xe is a rare and relatively expensive gas which can make it cost prohibitive for use.

It is thus, an object of the invention to provide an efficient method of purifying Xe from the patient's exhalations would allow recycle of this anaesthetic and decrease the usage cost in anaesthetic applications.

### SUMMARY

A method is disclosed for recovering and reusing Xenon from a patient's exhalations. It comprises the following steps. An Xe-containing inhalation gas is administered to a patient with a ventilator. Exhaled breath comprising CO₂, O₂, N₂, and Xe is directed from the patient to a feed side of a membrane where a permeate gas enriched in CO₂, O₂, and N₂ and depleted in Xe preferentially permeates through the membrane to a permeate side thereof, the membrane including a primary gas separation medium comprising a perfluorinated cyclic ether polymer. A residue gas enriched in Xe and depleted in CO₂, O₂, and N₂ is withdrawn from a residue port of the membrane. Makeup O₂ and makeup Xe are added to the residue gas to provide the inhalation gas mixture.

Another method is disclosed for recovering and reusing Xenon from a patient's exhalations. It comprises the following steps. An Xe-containing inhalation gas is administered to a patient with a ventilator. Exhaled breath comprising CO₂, O₂, N₂, and Xe is directed from the patient to a feed side of a polymeric membrane where a permeate gas enriched in CO₂, O₂, and N₂ and depleted in Xe preferentially permeates through the membrane to a permeate side thereof, the polymeric membrane having the properties of: a N₂ permeance>40 GPU [10⁻⁶ cm³ (STP)/cm²·s·cm(Hg)], a CO₂ permeance>250 GPU [10⁻⁶ cm³ (STP)/cm²·s·cm(Hg)], and a N₂/Xe selectivity>3 at ambient temperature/pressure conditions. A residue gas enriched in Xe and depleted in CO₂, O₂, and N₂ is withdrawn from a residue port of the polymeric membrane. Makeup O₂ and makeup Xe is added to the residue gas to provide the inhalation gas mixture.

Still another method is disclosed for recovering and reusing Xenon from a patient's exhalations. It comprises the following steps. A Xe-containing inhalation gas is administered to a patient with a ventilator. Exhaled breath comprising CO₂, O₂, N₂, and Xe is directed from the patient to a feed side of a first membrane where a first permeate gas enriched in CO₂, O₂, and N₂ and depleted in Xe preferentially permeates through the first membrane to a permeate side thereof, the first membrane including a primary gas separation medium comprising a perfluorinated cyclic ether polymer. A first residue gas enriched in Xe and depleted in CO₂, O₂, and N₂ is withdrawn from a residue port of the first membrane. The first permeate gas is directed from the permeate side of the first membrane to a feed side of a second membrane where a second permeate gas enriched in CO₂, O₂, and N₂ and depleted in Xe preferentially permeates through the second membrane to a permeate side thereof, the second membrane including a primary gas separation medium comprising a perfluorinated cyclic ether polymer. A second residue gas enriched in Xe and depleted in CO₂, O₂, and N₂ is withdrawn from a residue port of the second membrane. Makeup O₂, makeup Xe, and the first and second residue gases are combined to provide the inhalation gas mixture.

Yet still another method is disclosed of recovery Xe from a patient's exhalations. It comprises the following steps. A patient's exhalations are fed from a ventilator to a membrane where it is separated into a CO₂ and N₂ enriched permeate and a Xe-enriched residue, the membrane being made of polymers or copolymers based on perfluoro-2,2-dimethyl-1,3-dioxole. M makeup Xe and makeup O₂ are added to the Xe-enriched residue. The combined makeup Xe, makeup O₂, and Xe-enriched residue are directed to the ventilator.

A system is disclosed for recovering and reusing Xe from an Xe-containing exhalations of a patient. The system comprises: a ventilator, a membrane, a return tube, a source of makeup O₂ and makeup Xe, a microprocessor, and a gas analyzer. The ventilator is adapted and configured to administer an inhalation gas containing Xe to a patient and collect the patient's exhalations. The membrane is based on poly(perfluoro-2,2-dimethyl-1,3-dioxole) and has a feed side, a permeate side, and a residue port, the feed side being in fluid communication with the ventilator to receive the patient's exhalations comprising CO₂, N₂, O₂, and Xe, the membrane being adapted and configured to receive the patient's exhalations at the feed side and separate the patient's exhalations into a permeate gas enriched in CO₂, N₂, and O₂ and a residue gas enriched in Xe. The return tube is in fluid communication with the residue port. The source(s) of makeup O₂ and makeup Xe are in fluid communication with the return tube. The microprocessor is adapted to control addition of makeup O₂ and makeup Xe from the source(s) to a residue gas in the tube. The gas analyzer is adapted to measure levels of O₂ and Xe in the combined makeup O₂, makeup Xe, and residue gas, wherein the microprocessor's controlled addition of makeup O₂ and makeup Xe is based upon the levels of O₂ and Xe measured by the analyzer and predetermined desired levels of O₂ and Xe in the inhalation gas.

Any of the disclosed methods of the disclosed system may include one or more of the following aspects:
the method further comprises the step of measuring levels of Xe and O₂ in the combined makeup O₂, makeup Xe, and residue gas wherein said addition of makeup O₂ and makeup Xe is controlled based upon the measured levels of Xe and O₂.
the method, further comprises the step of adding makeup moisture to the residue gas.
the method further comprises the steps of measuring levels of moisture, Xe and O₂ in the combined makeup moisture, makeup O₂, makeup Xe, and residue gas wherein said addition of makeup moisture, makeup O₂ and makeup Xe is controlled based upon the measured levels of moisture, Xe and O₂.
the method further comprises the steps of:
   applying a vacuum to the permeate side; and
   adjusting pressures of the makeup O₂, the makeup Xe, and the level of the vacuum applied to the permeate side such that the combined makeup O₂, makeup Xe, and residue gas has a pressure at or near ambient.
the membrane comprises hollow conjugate fibers comprising a sheath made of the primary gas separation medium around a core.
the perfluorinated cyclic ether polymer is a homopolymer or copolymer of a perfluorinated dioxole or a homopolymer or copolymer of perfluoro(4-vinyloxy-1-butene).
the homopolymer or copolymer of a perfluorinated dioxole includes repeating units represented by the formula:

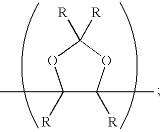

where each R is independently selected from the group consisting of F, a perfluoroalkyl group, and a perfluoroalkoxy group.
each R is independently selected from the group consisting of F, CF₃ and OCF₃.
the repeating units are represented by the formula:

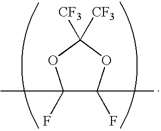

the perfluorinated cyclic ether polymer is a copolymer having repeating units represented by the formula:

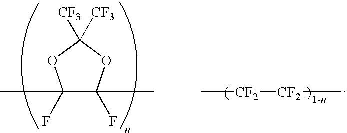

the repeating units are represented by the formula:

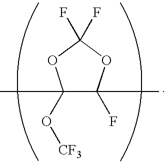

the perfluorinated cyclic ether polymer is a copolymer having repeating units represented by the formula:

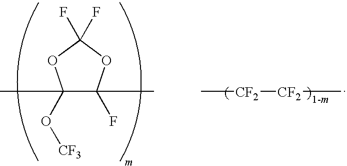

the homopolymer or copolymer of a perfluoro(4-vinyloxy-1-butene) includes repeating units represented by the formula:

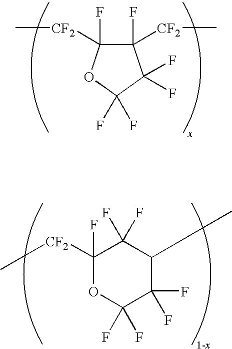

wherein:
   said source(s) includes makeup moisture;
   said microprocessor is adapted to control addition of moisture from said source(s) to the residue gas in said tube; and
   said microprocessor's controlled addition of makeup moisture is based upon the level of moisture measured by the analyzer and a predetermined desired level of moisture in the inhalation gas.
the system further comprises a vacuum in fluid communication with said permeate side.
the system further comprises a ballast container in fluid communication between said residue port and said ventilator.
the membrane comprises hollow conjugate fibers comprising a sheath made of the primary gas separation medium around a core.
the perfluorinated cyclic ether polymer is a homopolymer or copolymer of a perfluorinated dioxole or a homopolymer or copolymer of perfluoro(4-vinyloxy-1-butene).
the homopolymer or copolymer of a perfluorinated dioxole includes repeating units represented by the formula:

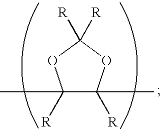

where each R is independently selected from the group consisting of F, a perfluoroalkyl group, and a perfluoroalkoxy group.
wherein each R is independently selected from the group consisting of F, CF₃ and OCF₃.
wherein the repeating units are represented by the formula:

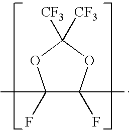

wherein the perfluorinated cyclic ether polymer is a copolymer having repeating units represented by the formula:

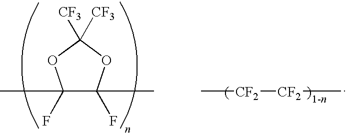

wherein the repeating units are represented by the formula:

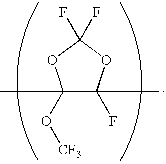

wherein the perfluorinated cyclic ether polymer is a copolymer having repeating units represented by the formula:

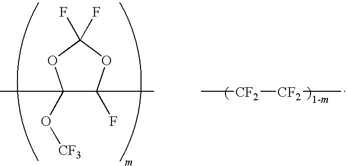

wherein the homopolymer or copolymer of a perfluoro(4-vinyloxy-1-butene) includes repeating units represented by the formula:

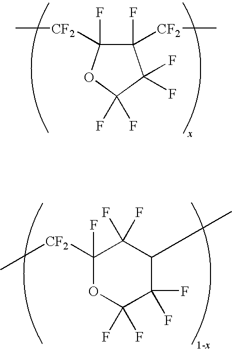

### BRIEF DESCRIPTION OF THE DRAWING

For a further understanding of the nature and objects of the present invention, reference should be made to the following detailed description, taken in conjunction with the accompanying drawing, wherein:

FIG. 1 illustrates one embodiment of a system for recovery and reuse of Xe from a patient's exhalations.

FIG. 2 illustrates another embodiment of a system for recovery and reuse of Xe from a patient's exhalations employing two membrane modules.

### DESCRIPTION OF PREFERRED EMBODIMENTS

A membrane is used to separate out N₂ and CO₂ from a patient's exhalations that also include Xe. The Xe residue gas is then supplemented with makeup Xe and makeup O₂ and directed back to a ventilator for administration to the patient.

The membrane of the invention should have a N₂ permeance>40 GPU [10⁻⁶ cm³ (STP)/cm²·s·cm(Hg)], a CO₂ permeance>250 GPU [10⁻⁶ cm³ (STP)/cm²·s·cm(Hg)], and a N₂/Xe selectivity>3 at ambient temperature/pressure conditions. The use of these relatively high permeance membranes allows the construction of reasonably sized devices which can remove the non-anesthetic gases at ambient feed pressures.

The membrane includes a primary gas separation medium. The membrane may be configured in a variety of ways: sheet, tube, hollow fiber, etc. In the case of a hollow fiber membrane, either a monolithic or conjugate configuration may be selected. If the monolithic configuration is selected, the primary gas separation medium is uniformly distributed throughout the fiber.

If the conjugate configuration is selected, while the primary gas separation medium present may be present either as a core beneath a sheath, preferably it is present as a sheath (in such a case the sheath is also called the selective layer) around a core. In this latter configuration, the core has an OD in the range of from about 100 and 2,000 μm, preferably from about 300 μm and 1,500 μm. The core wall thickness is in a range of from about 30 μm to 300 μm, preferably no greater than about 200 μm. The core inner diameter is from about 50 to 90% of its outer diameter. The selective layer is less than about 1 μm thick, preferably less than about 0.5 μm thick. Preferably, the thickness is in a range of from about 150 to 1,000 angstroms. More preferably, the thickness is in a range of from about 300 to 500 angstroms.

The core may be made of several different types of polymeric materials, including but not limited to polysulfones, ULTEM 1000, or a blend of ULTEM and a polymeric material available under the trade name MATRIMIDE 5218. Ultem 1000 is a polymer represented by Formula I below and is available from a variety of commercial sources, including Polymer Plastics Corp., Reno, Nev. or Modern Plastics, Bridgeport, Conn.).

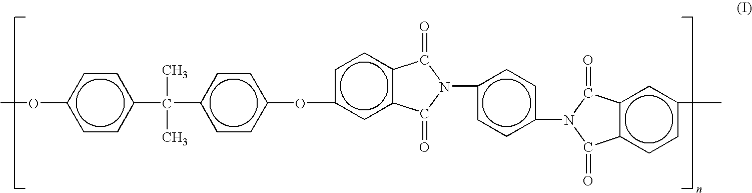

MATRIMID 5218 is the polymeric condensation product of 3,3′,4,4′-benzophenone tetracarboxylic dianhydride and 5(6)-amino-1-(4′-aminophenyl)-1,3,3′-trimethylindane, commercially available from Ciba Specialty Chemicals Corp.

Suitable materials for use as the primary gas separation medium include but are not limited to perfluorinated cyclic ether polymers. Preferred perfluorinated cyclic ether polymers include homopolymers or copolymers of perfluorinated dioxoles (Formula II) or polymers or copolymers of perfluoro(4-vinyloxy-1-butene) (Formula III or Formula IV). The primary gas separation medium of the membrane may also be a blend of one or more of the homopolymers and/or copolymers.

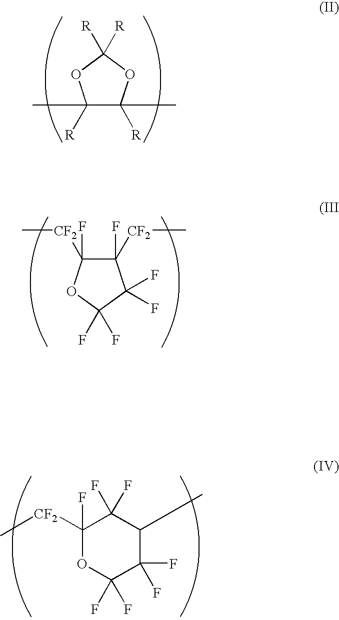

where each R is independently selected from the group consisting of F, a perfluoroalkyl group, and a perfluoroalkoxy group. A preferred perflouoroalkyl group is CF₃ and a preferred perfluoroalkoxy group is OCF₃. For homopolymers or copolymers including repeating units represented by Formula II, preferred examples include those represented by Formula IIa [poly(perfluoro-2,2-dimethyl-1,3-dioxole) with or without one or more other monomers] and lib [poly(2,2,4-trifluoro-5-trifluoromethoxy-1,3-dioxole) with or without one or more other monomers such as tetrafluoroethylene].

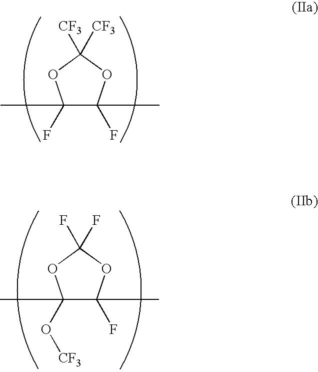

A preferred copolymer including repeating units of Formula IIb is represented by Formula V. When m is 0.6, such a copolymer is available from Solvay Solexis under the trade name Hyflon AD 60. When m is 0.8, such a copolymer is available from Solvay Solexis under the trade name Hyflon AD 80.

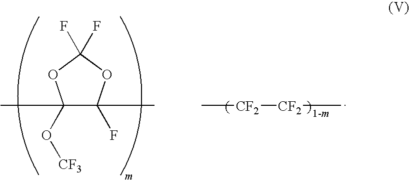

A preferred copolymer including repeating units of Formulae III and IV is represented by Formula VI. Such a copolymer is available from Asahi Glass Comp. under the trade name Cytop where x is 0.84.

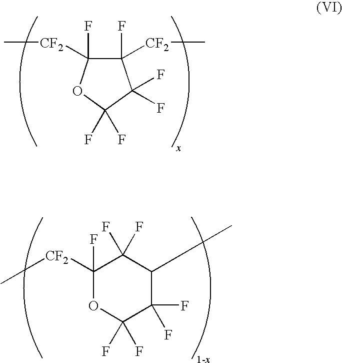

Most preferably, the perfluorinated cyclic ether polymer is a copolymer including repeating units of Formula IIa represented by Formula VII. When n is 0.87, such a copolymer is available from Dupont under the trade name Teflon AF2400. When n is 0.65, such a copolymer is available from Dupont under the trade name Teflon AF1600. This copolymer

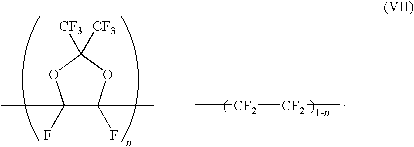

exhibits good selectivity for CO₂, O₂ and N₂ over Xe. This selectivity enables CO₂ and N₂ to be continuously and efficiently purged from the Xe containing exhaled stream thereby allowing this stream to be recycled back to the ventilator with small amounts of makeup Xe and O₂ (and optionally moisture). Consequently, the amount of Xe used in anaesthetic applications is decreased. The high permeance afforded by the use of this copolymer allows the patient to be ventilated with a recirculation loop that is entirely maintained at a pressure of 80−200 kPa, preferably 90−120 kPa, and most preferably near ambient pressures. Separation would be assisted with the use of a vacuum on the permeate side of the membrane.

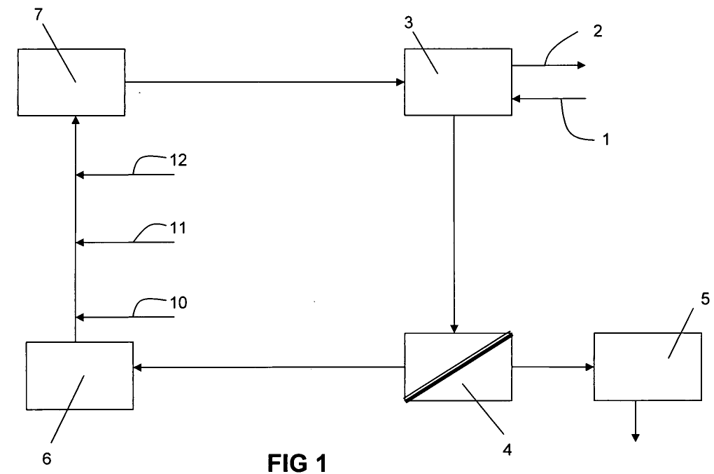

As best shown in FIG. 1, the exhaled stream 1 from a patient who is attached to a medical ventilator 3 operating at substantially ambient pressure is diverted to the feed side of a membrane module 4. The permeate side of the membrane module 4 is connected to a vacuum source 5 (such as vacuum pump) such that the ratio of the feed side pressure (such as 90-120 kPa) to that of permeate side pressure is >5:1. CO₂, H₂O, O₂, and N₂ preferentially permeate through the membrane 4 to the permeate side where they are vented. Xe is enriched in the residue gas which is directed to ballast container 6. A combination gas analyzer/microprocessor 7 controls the addition of makeup O₂ 10, optional makeup moisture 11, and make up Xe 12 (and any other makeup gases or vapor required for specific treatment in the gas mixture stream 2 to be inhaled) to the residue gas. The Xe-containing gas mixture with any makeup gases 10, 11, 12 is then directed back to ventilator 3 for administering to the patient via stream 2.

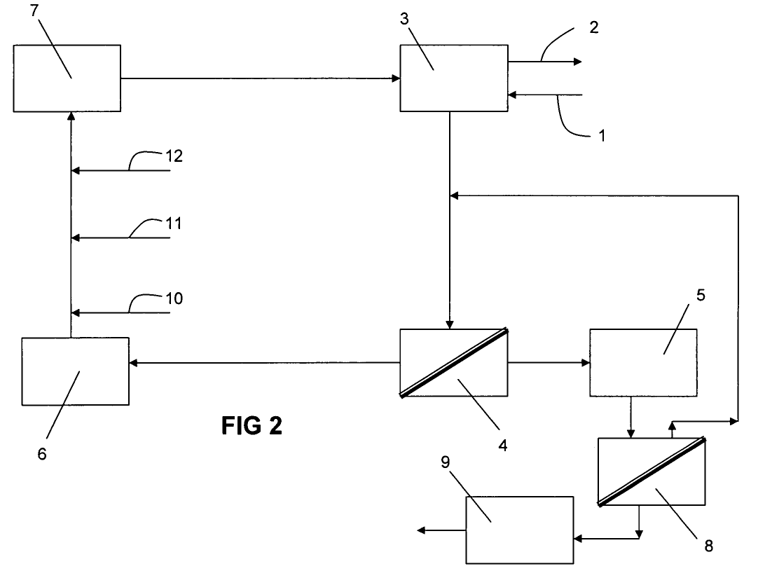

As best illustrated in FIG. 2, greater Xe recovery can be achieved by a two-stage membrane in comparison to the single-stage membrane of FIG. 1. The permeate discharged by the vacuum pump 5 evacuating the permeate side of the membrane module 4 can be fed to a similar second membrane module 8 plus second vacuum pump 9. The recovered Xe stream from membrane module 8 can be recycled back to the anesthetic recycle loop.

### EXAMPLE

A thin film of Teflon AF1600 was coated on a microporous polysulfone hollow fiber support by substantially the same procedure as taught in U.S. Pat. No. 6,540,813, the fiber-forming method disclosure of which is incorporated herein by reference. The coated fiber was potted into minipermeators and exposed to various pressurized pure gases at ambient temperature. The CO₂ permeance was determined to be 600-1000 GPU. The N2 permeance was 70-100 GPU. The selectivities (ratio of individual gas permeances) for various gases against Xe are shown in Table I:

TABLE I
Membrane Permeance

| | |
|---|---|
| CO₂/Xe | 36-40 |
| O₂/Xe | 9-10 |
| N₂/Xe | 4-7 |

Preferred processes and apparatus for practicing the present invention have been described. It will be understood and readily apparent to the skilled artisan that many changes and modifications may be made to the above-described embodiments without departing from the spirit and the scope of the present invention. The foregoing is illustrative only and that other embodiments of the integrated processes and apparatus may be employed without departing from the true scope of the invention defined in the following claims.

What is claimed is:

1. A method for recovering and reusing Xenon from a patient's exhalations, comprising the steps of:
administering a Xe-containing inhalation gas to a patient with a ventilator;
directing exhaled breath comprising CO₂, O₂, N₂, and Xe from the patient to a feed side of a membrane where a permeate gas enriched in CO₂, O₂, and N₂ and depleted in Xe preferentially permeates through the membrane to a permeate side thereof, the membrane including a primary gas separation medium comprising a perfluorinated cyclic ether polymer;
withdrawing a residue gas enriched in Xe and depleted in CO₂, O₂, and N₂ from a residue port of the membrane; and
adding makeup O₂ and makeup Xe to the residue gas to provide the inhalation gas mixture.

2. The method of claim 1, further comprising the step of measuring levels of Xe and O₂ in the combined makeup O₂, makeup Xe, and residue gas wherein said addition of makeup O₂ and makeup Xe is controlled based upon the measured levels of Xe and O₂.

3. The method of claim 1, further comprising the step of adding makeup moisture to the residue gas.

4. The method of claim 3, further comprising the step of measuring levels of moisture, Xe and O₂ in the combined makeup moisture, makeup O₂, makeup Xe, and residue gas wherein said addition of makeup moisture, makeup O₂ and makeup Xe is controlled based upon the measured levels of moisture, Xe and O₂.

5. The method of claim 1, further comprising the steps of:
applying a vacuum to the permeate side; and
adjusting pressures of the makeup O₂, the makeup Xe, and the level of the vacuum applied to the permeate side such that the combined makeup O₂, makeup Xe, and residue gas has a pressure at or near ambient.

6. The method of claim 1, wherein the membrane comprises hollow conjugate fibers comprising a sheath made of the primary gas separation medium around a core.

7. The method of claim 1, wherein the perfluorinated cyclic ether polymer is a homopolymer or copolymer of a perfluorinated dioxole or a homopolymer or copolymer of perfluoro(4-vinyloxy-1-butene).

8. The method of claim 7, wherein the homopolymer or copolymer of a perfluorinated dioxole includes repeating units represented by the formula: 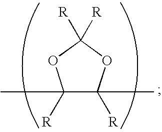
where each R is independently selected from the group consisting of F, a perfluoroalkyl group, and a perfluoroalkoxy group.

9. The method of claim 8, wherein each R is independently selected from the group consisting of F, CF₃ and OCF₃.

10. The method of claim 8, wherein the repeating units are represented by the formula:

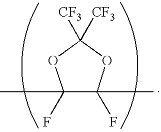

11. The method of claim 10, wherein the perfluorinated cyclic ether polymer is a copolymer having repeating units represented by the formula:

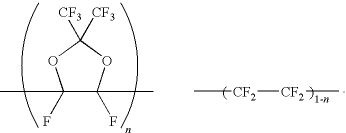

12. The method of claim 8, wherein the repeating units are represented by the formula:

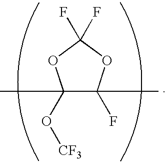

13. The method of claim 12, wherein the perfluorinated cyclic ether polymer is a copolymer having repeating units represented by the formula:

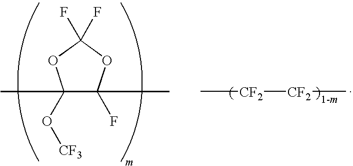

14. The method of claim 7, wherein the homopolymer or copolymer of a perfluoro(4-vinyloxy-1-butene) includes repeating units represented by the formula:

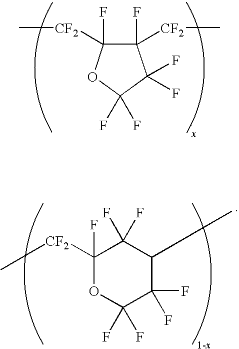

15. A method for recovering and reusing Xenon from a patient's exhalations, comprising the steps of:
administering a Xe-containing inhalation gas to a patient with a ventilator;
directing exhaled breath comprising CO₂, O₂, N₂, and Xe from the patient to a feed side of a polymeric membrane where a permeate gas enriched in CO₂, O₂, and N₂ and depleted in Xe preferentially permeates through the membrane to a permeate side thereof, the polymeric membrane having the properties of:
a N₂ permeance>40 GPU [10⁻⁶ cm³ (STP)/cm²·s·cm(Hg)],
a CO₂ permeance>250 GPU [10⁻⁶ cm³ (STP)/cm²·s·cm(Hg)], and
a N₂/Xe selectivity>3 at ambient temperature/pressure conditions;
withdrawing a residue gas enriched in Xe and depleted in CO₂, O₂, and N₂ from a residue port of the polymeric membrane; and
adding makeup O₂ and makeup Xe to the residue gas to provide the inhalation gas mixture.

16. A method for recovering and reusing Xenon from a patient's exhalations, comprising the steps of:
administering a Xe-containing inhalation gas to a patient with a ventilator;
directing exhaled breath comprising CO₂, O₂, N₂, and Xe from the patient to a feed side of a first membrane where a first permeate gas enriched in CO₂, O₂, and N₂ and depleted in Xe preferentially permeates through the first membrane to a permeate side thereof, the first membrane including a primary gas separation medium comprising a perfluorinated cyclic ether polymer;
withdrawing a first residue gas enriched in Xe and depleted in CO₂, O₂, and N₂ from a residue port of the first membrane;
directing the first permeate gas from the permeate side of the first membrane to a feed side of a second membrane where a second permeate gas enriched in CO₂, O₂, and N₂ and depleted in Xe preferentially permeates through the second membrane to a permeate side thereof, the second membrane including a primary gas separation medium comprising a perfluorinated cyclic ether polymer;
withdrawing a second residue gas enriched in Xe and depleted in CO₂, O₂, and N₂ from a residue port of the second membrane; and
combining makeup O₂, makeup Xe, and the first and second residue gases to provide the inhalation gas mixture.

17. A system for recovering and reusing Xe from an Xe-containing exhalations of a patient, comprising:
a ventilator adapted and configured to administer an inhalation gas containing Xe to a patient and collect the patient's exhalations;
a membrane based on poly(perfluoro-2,2-dimethyl-1,3-dioxole) having a feed side, a permeate side, and a residue port, said feed side being in fluid communication with said ventilator to receive the patient's exhalations comprising CO₂, N₂, O₂, and Xe, said membrane being adapted and configured to receive the patient's exhalations at said feed side and separate the patient's exhalations into a permeate gas enriched in CO₂, N₂, and O₂ and a residue gas enriched in Xe;
a return tube in fluid communication with said residue port;
a source(s) of makeup O₂ and makeup Xe in fluid communication with said return tube;
a microprocessor adapted to control addition of makeup O₂ and makeup Xe from said source(s) to a residue gas in said tube; and
a gas analyzer adapted to measure levels of O₂ and Xe in the combined makeup O₂, makeup Xe, and residue gas, wherein the microprocessor's controlled addition of makeup O₂ and makeup Xe is based upon the levels of O₂ and Xe measured by said analyzer and predetermined desired levels of O₂ and Xe in the inhalation gas.

18. The system of claim 17, wherein:
said source(s) includes makeup moisture;
said microprocessor is adapted to control addition of moisture from said source(s) to the residue gas in said tube; and
said microprocessor's controlled addition of makeup moisture is based upon the level of moisture measured by the analyzer and a predetermined desired level of moisture in the inhalation gas.

19. The system of claim 17, further comprising a vacuum in fluid communication with said permeate side.

20. The system of claim 17, further comprising a ballast container in fluid communication between said residue port and said ventilator.

21. The system of claim 17, wherein the membrane comprises hollow conjugate fibers comprising a sheath made of the primary gas separation medium around a core.

22. The system of claim 17, wherein the perfluorinated cyclic ether polymer is a homopolymer or copolymer of a perfluorinated dioxole or a homopolymer or copolymer of perfluoro(4-vinyloxy-1-butene).

23. The system of claim 22, wherein the homopolymer or copolymer of a perfluorinated dioxole includes repeating units represented by the formula: 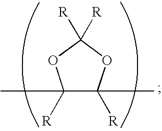
where each R is independently selected from the group consisting of F, a perfluoroalkyl group, and a perfluoroalkoxy group.

24. The system of claim 23, wherein each R is independently selected from the group consisting of F, CF₃ and OCF₃.

25. The system of claim 23, wherein the repeating units are represented by the formula:

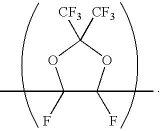

26. The system of claim 25, wherein the perfluorinated cyclic ether polymer is a copolymer having repeating units represented by the formula:

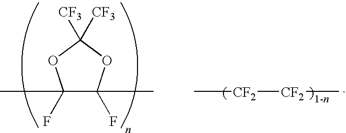

27. The system of claim 23, wherein the repeating units are represented by the formula:

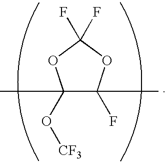

28. The system of claim 27, wherein the perfluorinated cyclic ether polymer is a copolymer having repeating units represented by the formula:

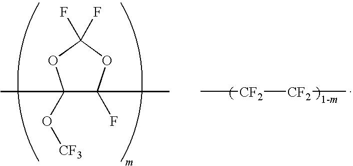

29. The system of claim 22, wherein the homopolymer or copolymer of a perfluoro(4-vinyloxy-1-butene) includes repeating units represented by the formula:

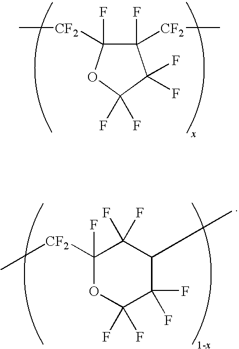

30. A method of recovery Xe from a patient's exhalations, comprising the steps of:
feeding a patient's exhalations from a ventilator to a membrane where it is separated into a CO₂ and N₂ enriched permeate and a Xe-enriched residue, the membrane being made of polymers or copolymers based on perfluoro-2,2-dimethyl-1,3-dioxole;
adding makeup Xe and makeup O₂ to the Xe-enriched residue; and
directing the combined makeup Xe, makeup O₂, and Xe-enriched residue to the ventilator.
